(19) United States
(12) Patent Application Publication
(10) Pub. No.: US 2007/0142805 A1
(43) Pub. Date: Jun. 21, 2007

(54) Disposable garment with angled fasteners and method for same

(75) Inventors: Paul Van Gompel, Hortonville, WI (US); LuAnn M. Beckman, Neenah, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/303,265

(22) Filed: Dec. 15, 2005

(51) Int. Cl. A61F 13/15

(52) U.S. Cl. 604/385.01; 604/389; 604/391

(57) ABSTRACT

A garment having a pair of front fasteners angled outwardly from the waist so the waist length is shorter than the hip length. In operation as a diaper, the garment may include a pair of similarly angled rear fasteners. In operation as a pant, the garment does include a pair of similarly angled fasteners. The angle at which to place the fasteners may be dependent on the crotch width of the garment.

### BACKGROUND OF THE INVENTION

This invention relates generally to a disposable garment for personal wear, and more particularly to such a disposable garment with improved fit and reduced leakage. Disposable garments are known for use in the manner of underpants for children and adults, garments worn like training pants for toddlers and garments worn like diapers for infants. Disposable absorbent garments are designed to absorb and contain bodily fluids but otherwise have a limited period of use before its ability to perform its intended function is exhausted. In particular, such disposable absorbent garments are intended to be discarded after one or more insults instead of being laundered. Typically, it is desirable for disposable garments to fit close to the body of the wearer's for comfort and discretion. Presently available disposable garments for wear about one's waist include a substantially liquid impermeable outer cover, a liquid permeable bodyside liner in a superposed relation with the outer cover, and an absorbent structure located between the outer cover and the bodyside liner for taking in and retaining liquid body exudates. The side edges of the garment form a pair of leg openings when the disposable garment is worn and the ends of the garment together form the waist opening thereof. Elastic components such as waist elastics, leg elastics and containment flaps are commonly used to enhance the fit and inhibit leakage from the garment.

With the advent of stretchable materials to construct disposable absorbent garments, various configurations for garments incorporating stretchable materials have been described. For example, once a stretchable material is selected to form a component of a garment, the material may be modified to provide a range of stretch characteristics. Simultaneous with the development of stretchable materials having lower cost and/or improved properties for use in disposable garments, developments with regard to the structural features of disposable garments have also occurred. An example of one such class of features is fasteners that can engage directly to the outer cover, liner, or inner surface of a disposable garment.

Even though significant and numerous advancements have occurred in the materials and structural features available for the construction of disposable garments, there remain opportunities for improvement and maintenance of the fit and containment capacity of such garments. For example, there remains a need for a disposable garment that provides improved fit and maintains fit of the garment on the wearer and which can be easily secured about the wearer.

### SUMMARY OF THE INVENTION

In one embodiment, a disposable absorbent garment generally has a chassis defining a longitudinal axis, a rear waist region located at the rear of the garment and having opposing rear intermediate edges, and a rear waist edge having a rear waist width. In addition the chassis has an opposite front waist region located at the front of the garment and having opposing front intermediate edges and a front waist edge having a front waist width. A crotch region defines a pair of opposed side edges having a lateral crotch width. There is a rear lateral hip width adjacent the rear waist region and a front lateral hip width adjacent the front waist region. A front pair of fasteners are attached to the front waist region, wherein the front pair of fasteners are angled with respect to the longitudinal axis of the garment.

In another aspect of the invention, the garment generally has a front pair of fasteners angled using the following method steps: determining an ideal body line for an average wearer of a predetermined garment size, by a) measuring a length of an ideal crotch side edge; and b) measuring a length of an actual crotch side edge of the garment. The angle is calculated from the length of the ideal crotch side edge and the length of the actual crotch side edge. In addition, the ideal body line may be partially defined by a subgluteal crease and an inguinal crease of the wearer, and further defined by a waist circumference of the wearer.

In yet another aspect of the invention, a diaper has a chassis defining a longitudinal axis, a rear waist region located at the rear of the garment and having opposing rear intermediate edges, and a rear waist edge having a rear waist width. In addition the chassis has an opposite front waist region located at the front of the garment and having opposing front intermediate edges and a front waist edge having a front waist width. A crotch region defines a pair of opposed side edges having a lateral crotch width. There is a rear lateral hip width adjacent the rear waist region and a front lateral hip width adjacent the front waist region. A front pair of fasteners are attached to the front waist region, wherein the front pair of fasteners are angled with respect to the longitudinal axis of the garment.

### BRIEF DESCRIPTION OF THE DRAWINGS

The foregoing and other features and aspects of the present invention and the manner of attaining them will become more apparent, and the invention itself will be better understood by reference to the following description, appended claims and accompanying drawings, where:

FIG. 1 illustrates a plan view of the bodyfacing surface of a representative disposable absorbent article incorporating a version of the improved fastening system;

FIG. 2 illustrates a plan view of the garment facing surface of the disposable absorbent article of FIG. 1;

FIG. 3 illustrates a plan view of the bodyfacing surface of another representative disposable absorbent article incorporating a version of the improved fastening system;

FIG. 4 illustrates a plan view of yet another embodiment of a disposable garment of the present invention in the form of a diaper illustrated in an unfolded and laid flat condition to show the surface of the diaper that contacts the skin of the wearer;

FIG. 5 illustrates a schematic perspective view of a simple garment corresponding to the ideal body line fit of a wearer;

FIG. 6 illustrates the garment of FIG. 5 in a flat, laid open condition; and

FIG. 7 illustrates a schematic plan view of a diaper showing the relationship between crotch width and leg perimeter.

Corresponding reference characters indicate corresponding parts throughout the several views of the drawings. However, the different embodiments may include different reference characters for similar parts.

### DETAILED DESCRIPTION OF THE INVENTION

The present invention is directed to providing disposable garments having an improved fastening system. Additionally, the present invention is directed to a garment having an unique combination of features that provide previously unrecognized and unexpected benefits. This detailed description of the present invention will include a description of a representative disposable garment including the various components of such garments. The description of the representative disposable garment will also include a description of many features encompassed by the present invention.

The present disclosure of the invention will be expressed in terms of its various components, elements, constructions, configurations, arrangements and other features that may also be individually or collectively be referenced by the term, “aspect(s)” of the invention, or other similar terms. It is contemplated that the various forms of the disclosed invention may incorporate one or more of its various features and aspects, and that such features and aspects may be employed in any desired, operative combination thereof. Moreover, the present invention may be applicable to non-disposable garments.

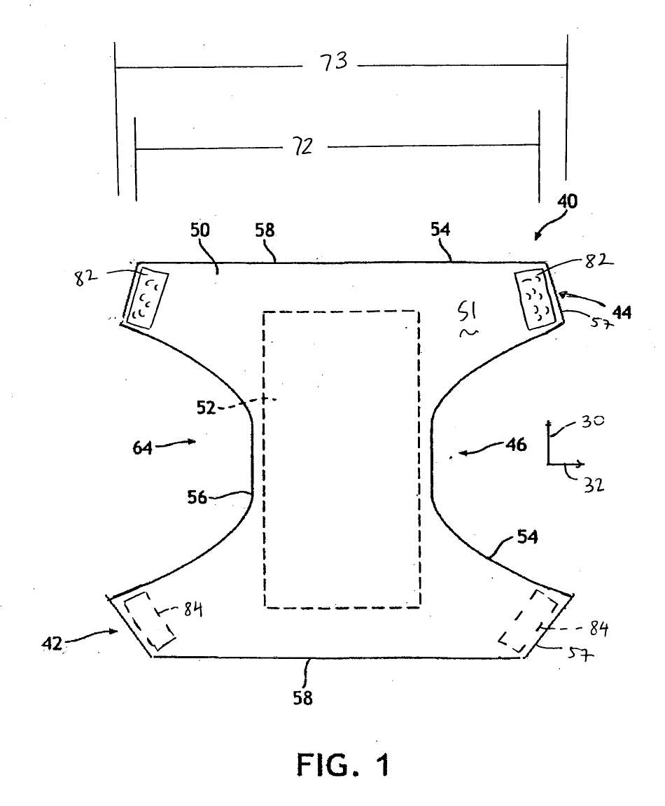

With reference to the drawings and in particular to FIGS. 1-4 a disposable garment according to one embodiment of the present invention is described herein with reference to a disposable absorbent garment, and more particularly to a diaper, generally indicated at 40 intended to be worn about the waist and lower torso of an infant or toddler. It is understood, however, that the various aspects of the present invention are equally adaptable to other types of disposable garments such as adult incontinence garments, training pants, disposable swim pants and feminine hygiene garments.

Disposable garments as referenced herein are intended for limited periods of use and are otherwise not intended for laundering. A disposable diaper, for example, is discarded after soiling by the wearer. Optionally, a disposable garment may include a replaceable absorbent insert wherein the remaining components of the garment may be reused several times before discarding. Differences between disposable garments and garments that are reused (e.g. underwear) include but are not limited to the following: 1) there is no need to launder a disposable item prior to first wear to remove any soiling due to the manufacturing process, excess dyes, sizing or other fabric treatments residual from the manufacturing process, and 2) disposable garments that contain superabsorbent materials cannot be washed without significantly altering the product to a non-usable state.

With regard to the designated surfaces of a disposable absorbent article and its components, the various upper or bodyfacing surfaces are configured to face toward the body of the wearer when the disposable absorbent article is worn by the wearer for ordinary use. The various opposing, lower or garment facing surfaces are configured to face away from the wearer's body when the disposable absorbent article is worn by the wearer.

As used herein, reference to two materials or elements being “joined” is intended to refer to the situation wherein the two materials or elements are 1) directly joined to one another, 2) where they are indirectly joined to one another, or 3) where they are indirectly joined to an intermediate element. Similarly, methods of joining two materials or elements include forming the elements or materials integrally, or attaching the elements together such as through the use of adhesive bonds, sonic bonds, thermal bonds, pinning, stitching, or a variety of other attachment techniques known in the art, as well as combinations thereof.

As used herein, the term “stretchable” refers to a material that may be extensible or elastomeric. That is, the material may be extended, deformed or the like, without breaking, and may or may not significantly retract after removal of an extending force. The terms “elastomeric” or “elastic” are used interchangeably herein and refer to that property of a material where upon removal of an elongating force, the material is capable of recovering to substantially its unstretched size and shape or the material exhibits a significant retractive force. The term “extensible” refers to that property of a material where upon removal of an elongating force, the material experiences a substantially permanent deformation or the material does not exhibit a significant retractive force. In particular, elastomeric materials utilized in connection with the present invention may be elongated/extended or stretched in at least one direction without breaking by at least 25% to at least 125% of its initial unstretched length in at least one direction, suitably by at least 50% to at least 150% of its initial unstretched length and which will recover, upon release of the applied stretching or biasing force, at least 10% of their elongation. It is generally suitable that the elastomeric material or composite be capable of being elongated by at least 100%, more suitably by at least 200%, of its relaxed length and recover at least 30% and more suitably 50% of its elongation upon release of a stretching, biasing force, within about one minute. Similarly, extensible or elongatable materials of the present invention may be capable of stretching in at least one direction without breaking by at least 25% to at least 125% of its initial unstretched length in at least one direction, suitably by at least 50% to at least 150% of its initial unstretched length, more suitably by at least 100% to at least 200% of its initial unstretched length. As an example, an extensible material having an initial unstretched length of 3 inches (7.6 centimeters) may be stretched without breaking to a stretched length of at least 3.75 inches (9.5 centimeters) in at least one direction for the “by at least 25%” value.

As used herein, the term “inboard” is intended to refer to the direction from an edge toward a respective centerline. The term “outboard” is intended to refer to a direction away from a respective centerline.

### REPRESENTATIVE EXAMPLES

FIG. 1 representatively illustrates one example of a disposable garment as generally indicated as garment 40. The dimensions of garment 40 can be such that it operates as a diaper or as a pant having side fasteners. The garment 40 defines a front waist region 42, a rear waist region 44, and a crotch region 46 which extends between and connects the front 42 and rear 44 waist regions. The front waist region 42 comprises the portion of the garment 40 which, when worn, is positioned on the front of the wearer, while the rear waist region 44 comprises the portion of the diaper which, when worn, is positioned on the back of the wearer. The crotch region 46 of the garment 40 comprises the portion of the diaper which, when worn, is positioned between the legs of the wearer and covers the lower torso of the wearer.

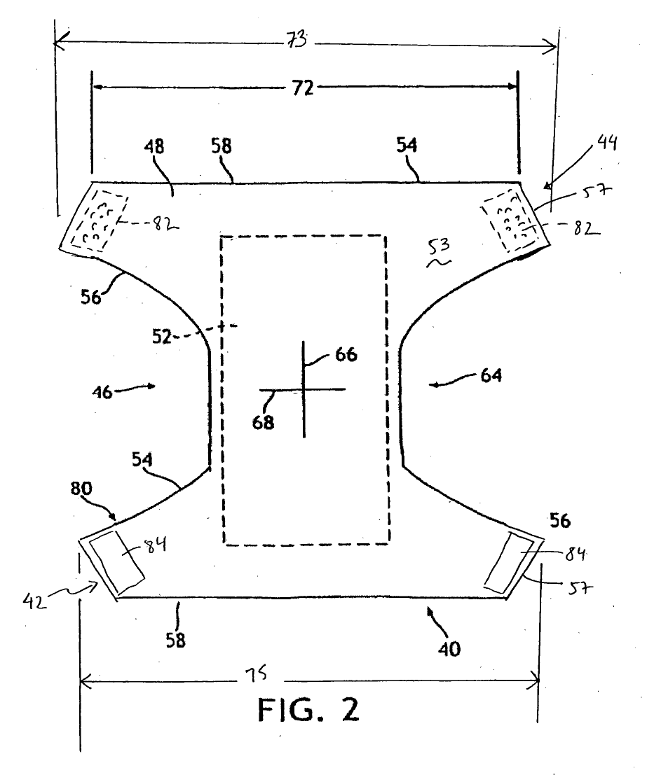

The several embodiments of garment 40 which are illustrated in FIGS. 1-3 include an outer cover 48 (FIG. 2), a bodyside liner 50 (FIG. 1), and an absorbent core 52 situated between the outer layer or outer cover 48 and the inner layer or liner 50. The outer edges of the garment 40 define a periphery 54 with laterally opposed, curvilinear or non-straight side edges 56 extending in the longitudinal direction, longitudinally opposed, laterally extending end edges 58, and intermediate side edges 57 located therebetween. The garment 40 may also include a system of elastomeric gathering members, such as leg elastics and waist elastics ( not illustrated). The longitudinal side edges 56 define leg openings 64 for the garment 40 that are curvilinear and/or contoured. The lateral end edges 58 are illustrated as straight, but optionally, may be curvilinear. The garment 40 additionally has a longitudinal centerline 66 and a lateral centerline 68. The garment 40 may also include additional components to assist in the acquisition, distribution and storage of bodily waste. For example, the garment 40 may include a transport layer, such as described in U.S. Pat. No. 4,798,603, issued to Meyer et al. attorney docket number 8,263, or a surge management layer as described herein.

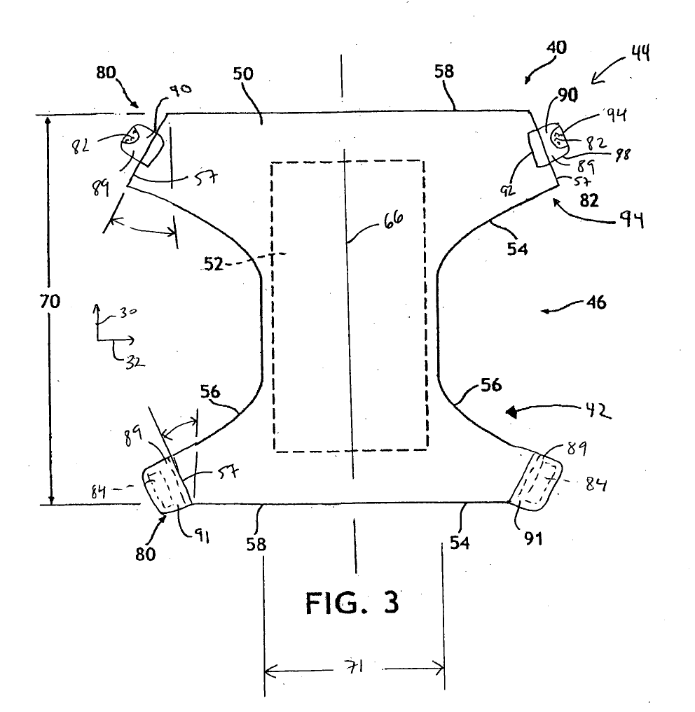

Referring to FIG. 3, the garment 40 generally has a longitudinal direction 30 that is perpendicular to a lateral direction 32 as indicated by the directional arrows provided in FIG. 3. The garment 40 defines a crotch width dimension 71 in lateral direction 32, and a length dimension 70 in the longitudinal direction 30. The diaper may have any desired shape, such as rectangular, I-shaped, a generally hourglass shape, or a T-shape.

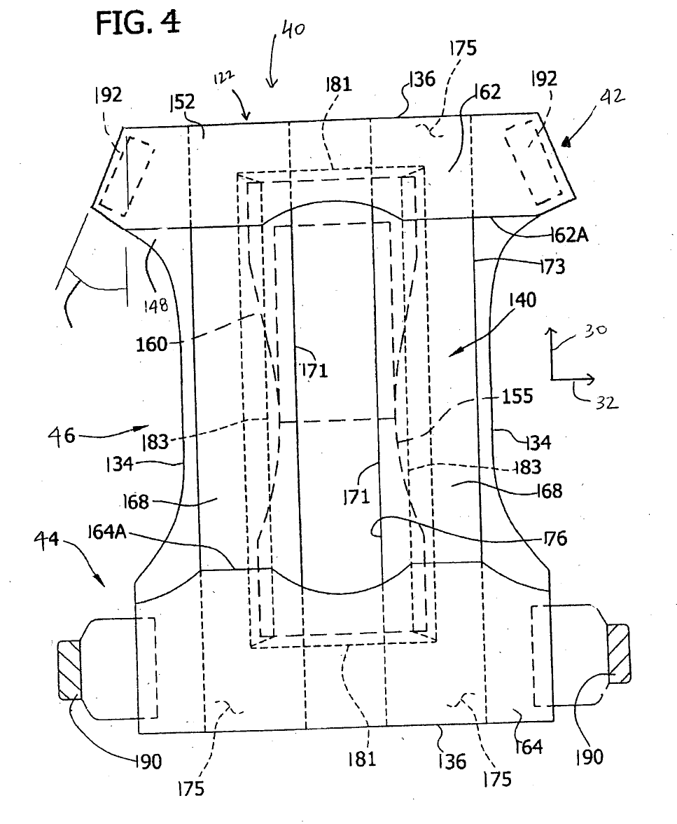

The outer cover 48 and the liner 50 may be generally coextensive e.g., FIGS. 1-3, or optionally, may be non-coextensive, e.g. FIG. 4. Either or both of the outer cover 48 and the liner 50 may have length and width dimensions which are generally larger than those of the absorbent core 52 and extend beyond the corresponding dimensions of the absorbent core 52 to provide longitudinal side edges 56 and lateral end edges 58 which may be connected or otherwise associated together in an operable manner.

The outer cover 48 may suitably be composed of various materials which are either liquid permeable or liquid impermeable. It is generally preferred that the outer cover 48 be formed from a material that is substantially impermeable to liquids. The outer cover 48 may be formed of a woven or nonwoven fibrous web layer which has been totally or partially constructed or treated to impart a desired level of liquid impermeability to selected regions that are adjacent or proximate the absorbent core 52.

Still further, the outer cover 48 may optionally be composed of a micro-porous “breathable” material which permits vapors to escape from the absorbent core 52 while still preventing liquid exudates from passing through the outer cover 48. Desirably, the outer cover 48 is made of a stretchable material. In a particular aspect, the outer cover 48 is made of an elastomeric material stretchable in one or more directions. In an alternative aspect, the outer cover 48 is made of an extensible material. Suitably, the outer cover 48 may be constructed from the particular materials cited in commonly owned U.S. Pat. Ser. No. 10/954536, file Sep. 29, 2004, incorporated by reference to the extent it is consistent with the present invention.

The liner 50 suitably presents a body-facing surface which is compliant, soft feeling, and non-irritating to the wearer's skin. Further, the liner 50 may be less hydrophilic than the absorbent core 52, to present a relatively dry surface to the wearer, and is sufficiently porous to be liquid permeable, permitting liquid to readily penetrate through its thickness. A suitable liner 50 may be manufactured from a wide selection of web materials, such as porous foams, reticulated foams, apertured plastic films, natural or synthetic fibers for example, polyester or polypropylene fibers, or a combination of natural and synthetic fibers. The liner 50 is suitably employed to help isolate the wearer's skin from liquids held in the absorbent core 50.

Desirably the liner 50 is made of a stretchable material. In a particular aspect, the liner 50 is made of an elastomeric material. Suitable elastomeric materials are stretchable in one or more directions. Suitable elastomeric materials for construction of the liner 50 can include elastic strands, LYCRA elastics, cast or blown elastic films, nonwoven elastic webs, meltblown or spunbond elastomeric fibrous webs, as well as combinations thereof. In an alternative aspect, the liner 50 is made of an extensible material.

The liner material 50 may be treated with a surfactant. This can be accomplished by a variety of techniques known to those skilled in the art. Treating the liner material 50 with a surfactant generally renders the liner material 50 more hydrophilic. This typically results in liquid penetrating the liner material 50 faster than if it were not treated.

An absorbent assembly may include at least an absorbent core 52. Absorbent core 52 may include a matrix of hydrophilic fibers, such as a web of cellulosic fluff, mixed with particles of a high-absorbency material commonly known as superabsorbent material.

In a particular version, the absorbent core 52 includes a mixture of superabsorbent hydrogel-forming particles and wood pulp fluff. The wood pulp fluff may be exchanged with synthetic polymeric, meltblown fibers or with a combination of meltblown fibers and natural fibers. The superabsorbent particles may be substantially homogeneously mixed with the hydrophilic fibers or may be non-uniformly mixed.

The absorbent core 52 may have any of a number of shapes. For example, the absorbent core 52 may be rectangular, I-shaped or T-shaped. It is often desired that the absorbent core 52 be narrower in the crotch portion than the rear or front portions.

The high-absorbency material can be selected from natural, synthetic and modified natural polymers and materials. The high-absorbency materials can be inorganic materials, such as silica gels, or organic compounds, such as crosslinked polymers. Further, the high-absorbency material may be in a variety of geometric forms. It is desired that the high-absorbency material be in the form of discrete particles. However, the high-absorbency material may also be in the form of fibers, flakes, rods, spheres, needles, or the like. Often, the high-absorbency material is present in the absorbent core 52 in an amount of from about 5 to about 100 weight percent based on total weight of the absorbent core 52.

Referring again to FIGS. 1-3, illustrated are versions of a garment 40 in its generally flat-out, uncontracted state i.e., with all elastic induced gathering and contraction removed. The garment 40 includes a liner 50 and an outer cover 48 which are coextensive and have length and width dimensions generally larger than those of an absorbent core 52. The liner 50 is associated with and superposed on the outer cover 48 to thereby form the periphery 54 of the garment 40. The periphery 54 defines an outer perimeter or edges of the garment 40. The periphery 54 generally includes longitudinal side edges 56, lateral end edges 58, and intermediate edges 57.

The disposable absorbent articles described herein include a fastening system 80 for securing the absorbent article about the waist of the wearer. The illustrated versions of the garment 40 include such a fastening system 80. If garment 40 is used as a diaper, the fastening system includes at least two front fasteners 84 and optionally, at least two rear fasteners 82. The rear fasteners are used primarily to adjust fit, not as the primary fasteners that retain the garment on the wearer. If garment 40 is used as a pant, the fastening system includes at least both the two front fasteners 84 and the two rear fasteners 82.

In operation as a diaper, front fasteners 84 provide improved securement of the garment 40 about the waist of the wearer. For example, as representatively illustrated in FIGS. 1-3, the garment 40 includes at least two front fasteners 84 situated in the front waist region 42 of the garment, and located adjacent each intermediate edge 57. The front fasteners 84 are configured to encircle the hips of the wearer and engage the bodyfacing surface of the liner 50 in the rear waist region 44 of the garment 40. Suitably, as representatively illustrated in FIGS. 1-2, the front fasteners 84 are hook type fasteners which are releasably engageable directly with the bodyfacing surface 51 of the liner 50.

Alternatively, the garment 40 may include one or more fastening panels not illustrated on the bodyfacing surface 51 of the rear waist region 44 to which the front fasteners 84 are releasably engageable.

In operation as a diaper, the optional rear fasteners 82 are situated in the rear waist region 44 of the garment 40, and may be located inboard each intermediate edge 57.

The rear fasteners 82 are configured engage the outer cover 48 of the front waist region 42 of the garment 40 for holding the diaper on the wearer. Desirably, the rear fasteners 82 are releasably engageable directly with the garment facing surface 53 of the outer cover 48. The garment facing surface 53 may include a fastening panel (not illustrated) situated in the front waist region 42 on the outer cover 48. In such a configuration, the rear fasteners 82 are releasably engageable with the fastening panel to maintain the garment 40 about the waist of the wearer. As representatively illustrated in FIGS. 1 and 3, the rear fasteners 82 may be hook type fasteners and the outer cover 48 or fastening panel may be configured to function as a complimentary loop type fastener. Desirably, the rear fasteners 82 are hook type fasteners which are releasably engageable with the outer cover 48. Such an arrangement provides the ability to vary the size of the waist opening in very small increments over a wide range to fit the waist of the wearer. The rear fasteners 82 may have a variety of shapes and sizes which provide the desired fastening of the garment 40 about the waist of the wearer.

In operation as a pant, fastener 82 and fastener 84 may overlap and engage one another to form a side seam. Of course, fastener 82 may attach directly to the outer cover garment facing surface 53.

Regardless of how garment 40 is used, the fastening system 80 may be presented to a user in a flat configuration, such that the user may don the garment without removal of clothes. Alternatively, the fastening system 80 may be presented to a user in a prefastened configuration, such that the user may don the garment 40 as one would don underpants.

An alternative fastening system 80 may include one or more ears 89. Ears 89 may be included in the front waist region 42, the rear waist region 44 or both the front and rear waist regions 42, 44. As illustrated in FIG. 3, each ear 89 includes a proximal edge 92, an opposed distal edge 94, a first connecting edge 96, and a second connecting edge 98.

As used herein, the proximal edge 92 is that edge of the ear 89 which is joined to a intermediate edge 57 of the garment 40. The distal edge 94 is that edge of the ear 89 which is opposite the proximal edge 92 moving in a direction outboard from a longitudinal centerline 66 of the garment 40. The first 96 and second 98 connecting edges connect the proximal edge 92 and the distal edge 94 thereby defining a body of material which at least partially defines an ear 89. The ear 89 may be made of a variety of materials including those that are extensible, non-extensible, elastomeric and/or non-elastomeric. Desirably, the ear 89 is made of a non-elastomeric material.

The garment 40 illustrated in FIG. 3 may be used as a diaper and includes a version of the alternative fastening system 80 described herein. Specifically, the fastening system 80 of FIG. 3 includes at least two rear fasteners 82 and at least two front fasteners 84.

Typically, the rear fasteners 82 are located inboard of a distal edge 94 on a bodyfacing surface of a rear ear 90. At least a portion of the proximal edge 92 of the rear ear 90 is joined to the rear waist region 44 of the garment 40, with the distal edge 94 of the ear extending outboard from the longitudinal centerline 66 and forming a portion of the side edge 56 of the diaper. Desirably, the rear fasteners 82 are releasably engageable directly with the garment facing surface of the outer cover 48. Alternatively, the garment 40 may include a fastening panel (not illustrated) situated in the front waist region 42 of the garment facing surface of the outer cover 48. In such a configuration, the rear fasteners 82 are releasably engageable with the fastening panel to maintain the garment 40 about the waist of the wearer.

Still referring to FIG. 3, the front fasteners 84 are located inboard of a distal edge 94 on a garment facing surface of a front ear 91. At least a portion of the proximal edge 92 of the front ear 91 is joined to the front waist region 42 of the garment 40, with the distal edge 94 of the ear extending outboard from the intermediate edge 57 of the diaper. Desirably, the front fasteners 84 are hook type fasteners which are releasably engageable directly with the bodyfacing surface of the liner 50. Alternatively, the garment 40 may include one or more fastening panels or waist stretch panels (not illustrated) on the bodyfacing surface 5 1 of the rear waist region 44 to which the front fasteners 84 are releasably engageable.

Suitably, at least the front fasteners 84 and optionally the front ears 91 of fastening system 80 are positioned at an angle. The purpose of positioning at least the front fasteners 84 at an angle with respect to longitudinal direction 66 is to provide enhanced fit. The angle maybe determined using a method based upon certain measurements taken from the idealized “body line fit” of an average wearer for a given diaper or pant size as described by way of example, herein. “Body line fit” as used herein refers to an area of coverage for a pant or diaper, defined by a full waist band at a wearer's navel to the small of the wearer's back, and a circumference of each leg which follows both the inguinal crease and the sub-gluteal crease. FIG. 6 shows the outline of a disposable absorbent garment 40(i) in a flat, laid-open condition, which is designed to fit to a wearer's body line. Fig, 5 shows the garment 40(i) in a fastened condition, as if it were fastened about a wearer.

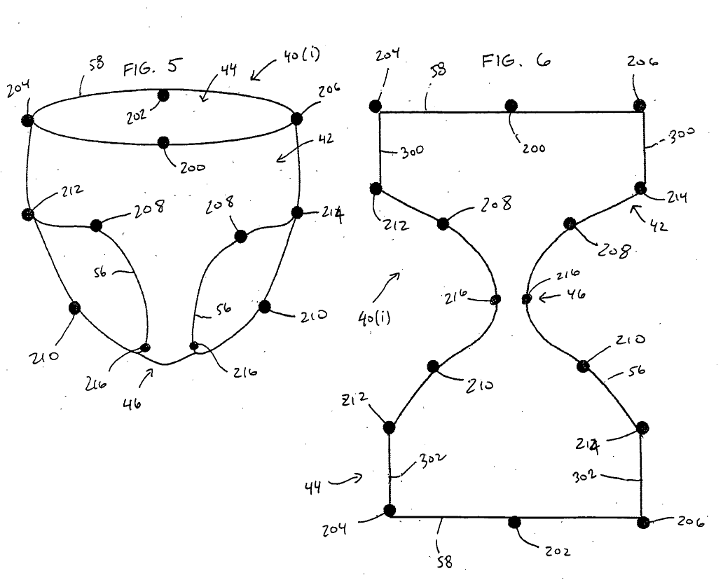

The garment 40(i) in FIGS. 5-6 demonstrates a minimal surface area for coverage, and zero volume for receiving exudates. Certain points are indicated on garment 40(i) which correspond to particular anatomical features. Referring to FIG. 6, a point 200 is centrally located on the end edge 58 of the front waist region 42, and corresponds to the wearer's navel. A point 202 similarly situated on the back waist region 44 corresponds to the small of the back. On the wearer's right and left sides of the garment are points 204, 206 respectively. Points 204 and 206 define the distal ends of each end edge 58. Thus, end edges 58 are positioned at the waist line, and likewise, points 200, 202, 204, and 206 are positioned at the waist. Each crotch side edge 56 has four points that coincide with a part of the wearer's body. Specifically, on the front waist region 42 are points 208 which correspond to about the center of the front thigh at the inguinal crease. On the back waist region 44 are points 210 which correspond to a point on about the center of the wearer's sub-gluteal crease. On the wearer's right and left sides of the garment are points 212, 214 where the front inguinal crease line aligns with the back subgluteal crease, respectively. Points 212 and 214 define the distal ends of each side edge 56. Points 216 are located at opposite sides of the crotch at substantially the narrowest width of the crotch region 46. Thus, side edges 56 are positioned at the leg line generally defined by the inguinal crease and the sub-gluteal crease curving inward toward the crotch; likewise, points 208-216 are positioned about the leg opening of the garment 40(i).

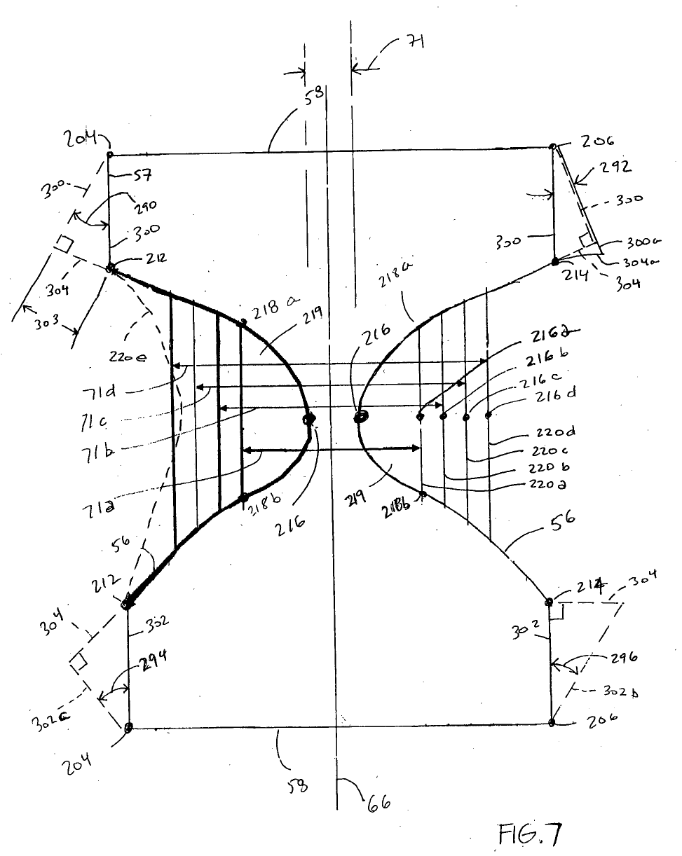

To fit a wider range of wearers for a given size, and to accommodate absorbent materials for receiving bodily exudates, at least the crotch width 71 is increased from the ideal body line. Referring now to FIG. 7, increasing the width of the crotch region 46 creates the desired volume. One possible way to widen crotch region 46 is to extend the crotch outboard from the ideal crotch width 71. This creates an additional crotch area 219 outboard of points 216. In a first example, each crotch area 219 may be defined by the portion of side edges 56 that is truncated by an edge 220(a). Edge 220(a) may be substantially parallel to longitudinal centerline 66. Of course, the edge 220(a) may be non-parallel, and may have a convex or concave curvature, if desired. The intersection of edge 220(a) with sides edges 56 is noted by points 218(a) and 218(b).

Various other crotch widths may be achieved from the ideal body line as demonstrated by optional other edges 216(b)-(d); these edges are demonstrative only and one skilled in the art will realize that any width between points 204 and 206 is possible. The first demonstrated width is measured between each point 216, e.g. 216(a), and referenced as corresponding width 71(a). Other demonstrated widths are referenced as 71(b)-(d). Of course, the edge 220 may have a different curvilinear shape, and is not intended to be limited to a straight line, see phantom edge 220(e). The width of crotch region 46 is dependent upon various factors, including but not limited to the thickness of the absorbent core. A commonly owned invention, described in U.S. Publication 2005/0148976, printed Jul. 7, 2005, further describes factors used to determine other fit aspects of an absorbent article, and is incorporated by reference to the extent that it consistent with the present invention.

Referring still to FIG. 7, when the crotch width 71 is increased from the body line fit, it is suitable to set front sides edges 300 and rear side edges 302 at an angle to create a platform for an angled fastener, e.g. fasteners 82 and 84 as seen in FIG. 2. This allows the fasteners to be positioned at an angle. The purpose of setting front and rear side edges at an angle is to maintain the waist width or longitudinal edge width 72 and increase the hip width 73 at front waist region 42, and optionally, maintain the waist width or longitudinal edge width 72 and increase the hip width 73 at back waist region 44 (references 72, 73 are shown at top of FIG. 2). Thus, the increased amount of material between the wearer's legs will not put as much strain on the fasteners if they are set at an angle as opposed to being oriented parallel with respect to the longitudinal centerline 66.

There are several ways to achieve angled side edges 300 and 302. One skilled in the art will realize that the fastener angle is what determines fit, thus, intermediate edges 57 do not have to be at the same angle as the fasteners. In a first embodiment as seen in the upper left-hand quadrant of FIG. 7, front intermediate edge 300 maintains a substantially constant length and is rotated outwardly from waist point 204 at an angle 290. The angle 290 is determined by the length 303 of additional edge 56, referenced as portion 304, shown in phantom. In turn, the length 303 is determined as follows: (a) measure the starting length of edge 56 when the crotch width is 71, this representing the ideal body line fit (hereinafter referred to as “P1”); (b) measure the new length of edge 56 when the crotch is increased, for example to width 71(a), the actual fit (hereinafter referred to as “P2”). If the absorbent article is a diaper as shown in FIGS. 1-4, length 303 is equal to the difference of P2 from P1. If the absorbent article is a used as a pant that has mating side fasteners, the length may be the difference of P2 from P1, divided by 2.

This method of angling a intermediate edge for a pant also applies to rear intermediate edge 302.

Results of the above technique as applied to an ideal body line for a Step 3 sized pant is shown in thee following table, Table 1.

TABLE 1
Kimberly-Clark Step 3 Pant

| Actual crotch width (mm) | Leg Perimeter (mm) | Angle (degrees) |
|---|---|---|
| 26 (ideal) | 305 (ideal, P1) | 0 |
| 100 | 273 (P2) | 14 |
| 127 | 262 (P2) | 20 |
| 152 | 250 (P2) | 25 |
| 177 | 231 (P2) | 33 |

In a second embodiment as seen in the upper right-hand quadrant in FIG. 7, front intermediate edge 300 is lengthened as it is rotated outwardly from point 206 at an angle 292. The new intermediate edge is referenced as intermediate 300(a). The corresponding additional portion 304a may be curved as shown, or straight. The length 304 (shown in phantom) is determined as in the first embodiment. The length of 304(a) is determined trigonometrically by assuming 304(a) to be a straight line. This method of angling a intermediate edge also applies to a rear intermediate edge 302.

In a third embodiment as seen in the lower left-hand quadrant FIG. 7, intermediate edge 302 is reduced in length as it is rotated outwardly from point 204 at an angle 294. The new intermediate edge is referenced as intermediate 302(c). The corresponding additional portion 304 may be straight as shown, or curved. The length 304 (shown in phantom) is determined as in the first embodiment. The length of 304(a) is then determined trigonometrically by assuming 304(a) to be a straight line. This method of angling a intermediate edge also applies to front intermediate edge 300.

In a fourth embodiment as seen in the lower right-hand quadrant of FIG. 7, intermediate edge 300 is increased in length as it is rotated outwardly from point 206 at an angle 296. The additional portion 304 is perpendicularly oriented with respect to longitudinal centerline 66. The length of additional portion 304 (shown in phantom) is determined as in the first embodiment. This method of angling a intermediate edge also applies to front intermediate edge 300.

When garment 40 is a diaper, it is noted that the rear fasteners 84 may be omitted or not be angled with respect to the longitudinal centerline 66. The angling of these rear fasteners may not be as critical in this case because the side seam of the garment is formed between the liner 50 and the front fasteners 84, as described herein.

The variety of embodiments shown in the four quadrants of FIG. 7 are for example only. It is intended that each of the separate embodiments would be used separately, or at least symmetrically with respect to the longitudinal centerline 66. One skilled in the art will realize that there may be variations to the shape of the angled portion, or that there may be ears 190 and/or tabs added to the angled portion as seen in FIG. 4.

Suitable fasteners are well known to those of skill in the art and can include adhesive tape tab fasteners, hook and loop fasteners, mushroom fasteners, snaps, pin, belts and the like, and combinations thereof. For example, as representatively illustrated in FIGS. 1 and 3, the rear fasteners 82 may be hook type fasteners and the outer cover 48 or fastening panel not illustrated may be configured to function as a complimentary loop type fastener. Desirably, the rear fasteners 82 are hook type fasteners which are releasably engageable with at least a portion of an outer cover 48. The front fasteners 84 may also be hook type fasteners and the liner 50 or fastening panel (not illustrated) may be configured to function as a complimentary loop type fastener. Desirably, the front fasteners 84 are hook type fasteners which are releasably engageable with at least a portion of a liner 50. Such an arrangement provides the ability to vary the size of the waist opening in very small increments over a wide range to fit the waist of the wearer. The first 82 and second 84 fasteners may have a variety of shapes and sizes which provide the desired fastening of the diaper about the waist of the wearer.

In one embodiment, a disposable absorbent article has opposed longitudinal side edges 56, opposed lateral end edges 58, a longitudinal centerline 66, a front waist region 42, a rear waist region 44 and a crotch region 46 which extends between and connects the front waist region and the rear waist region. The disposable garment includes a bodyfacing surface, a garment facing surface and a fastening system 80. The disposable garment may include an outer cover 48 having a bodyfacing surface and a garment facing surface. The outer cover 48 may comprise a stretchable material. Alternatively, the outer cover 48 may comprise an elastomeric material. The disposable garment may include a liner 50 having a bodyfacing surface and a garment facing surface, and being superposed over the bodyfacing surface of the outer cover 48. The liner 50 may comprise a stretchable material. Alternatively, the liner 50 may comprise an extensible material. An absorbent core 52 may be disposed between the garment facing surface of the liner 50 and the bodyfacing surface of the outer cover 48. The fastening system 80 includes at least two rear fasteners 82 and two front fasteners 84. Each of the rear fasteners 82 is configured to releasably engage at least a portion of the garment facing surface. At least a portion of each second fastener 84 is located in the front waist region 42 and situated at an angle and inboard from each longitudinal side edge 56 of the front waist region. Each of the front fasteners 84 is configured to releasably engage at least a portion of the bodyfacing surface. The front waist region 42 has a hip width 73 extending between opposed longitudinal side edges 56 and a shorter waist width 72. Likewise, the rear waist region 44 may have a hip width extending between opposed longitudinal side edges 56, and a shorter waist width. The width of the front waist region 42 may be less than or the same as the width of the rear waist region 44.

Another example of a disposable garment that is suitable for the fastening system of the present invention is disclosed in FIG. 4. The materials from which this embodiment may be constructed are disclosed in previously incorporated application, U.S. Ser. No. 10/954536. Here, the disposable garment 40 of the illustrated embodiment generally has a front waist region 42, a back waist region 44, and a center region, otherwise referred to herein as a crotch region 46, extending longitudinally between

With further reference to FIG. 4, the garment 40 is a diaper that has a pair of laterally opposite side edges 134, and a pair of longitudinally opposite ends 136. The garment 40 may be of various suitable shapes when unfolded and laid flat. For example, the garment 40 may have an overall rectangular shape, T-shape, hour-glass shape or a general I-shape as in the illustrated embodiments. Examples of diaper configurations suitable for use in connection with the instant application are described in U.S. Pat. Nos. 4,798,603 issued Jan. 17, 1989 to Meyer et al.; 5,176,668 issued Jan. 5, 1993, to Bemardin; 5,176,672 issued Jan. 5, 1993 to Bruemmer et al.; 5,192,606 issued Mar. 9, 1993 to Proxmire et al.; and 5,509,915 issued Apr. 23, 1996 to Hanson et al, the disclosures of which are hereby incorporated by reference to the extent they are consistent with the present invention. Referring to FIG. 4, the illustrated garment 40 suitably comprises an inner layer, generally indicated at 140, an outer layer, generally indicated at 148 in generally opposed and more suitably superposed relationship with the inner layer, and an absorbent assembly, generally indicated at 160, disposed between the inner and outer layers of the diaper for taking in and retaining body exudates released by the wearer. The inner layer 140 has an inner, or body facing surface that at least in part defines the inner surface 162 of the garment 40.

The inner layer 140 of the illustrated embodiment of FIG. 4 may be a multiple component construction, and in particular comprises a front waist panel 162 broadly, a first end panel of the inner layer at least in part defining the front waist region 42 of the garment 40 and a back waist panel 164 broadly a second end panel of the inner layer at least in part defining the rear waist region 44 of the diaper. The front and back waist panels 162, 164 are suitably longitudinally spaced from each other. For example, the front waist panel has a longitudinally outer end coterminous with the outer layer 148 to define a longitudinal end 136 of the garment 40, and a longitudinally inner end 162A. The back waist panel 164 has a longitudinally outer end coterminous with the opposite end of the outer layer to define the opposite longitudinal end 136 of the garment 40, and a longitudinally inner end 164A spaced longitudinally from the inner end 162A of the front waist panel so that the spacing therebetween at least in part defines the crotch region 46 of the diaper.

The inner layer 140 further comprises a pair of laterally spaced side panels 168 that extend longitudinally through the crotch region 46 of the garment 40, and more suitably extend from the front waist region 42 through the crotch region to the back waist region 44 of the diaper, and ever more suitably from one end 136 of the garment 40 to the other end. Thus, it will be seen that the front and back waist panels 162, 164 and laterally spaced side panels 168 together define a central opening 176 of the inner layer 140 of the garment 40 through which liquid, semi-liquid and solid exudates released by the wearer pass to the absorbent assembly 160. Each of the side panels 168 has a laterally inboard edge 171 and a laterally outboard edge 173. In the illustrated embodiment, the side edges of the front and back waist panels 162, 164 and the side edges of the outer layer 140 are coterminous and together define the lateral side edges 134 of the garment 40 while the longitudinally inner ends 162A, 164A of the front and back waist panels 162, 164 and laterally inboard edges 171 of the side panels 168 together define the central opening 176 of the inner layer 140 of the diaper.

The front and back waist panels 162, 164 of the inner layer 140 are suitably compliant, soft feeling, and nonirritating to the wearer's skin. The waist panels 162, 164 may have any suitable shape, such as rectangular e.g., the front waist panel of the illustrated embodiment, trapezoidal or otherwise non-rectangular e.g., the back waist panel of the illustrated embodiment depending on the desired overall shape of the garment 40. It is understood, however, that the side edges of the front and back waist panels 162, 164 may instead be coterminous with the laterally outboard edges 173 of the connecting members 168 and remain within the scope of this invention.

The front and back waist panels 162, 164 may be suitably constructed from the materials described in U.S. Ser. No. 10/954536, previously incorporated herein.

The absorbent assembly 160 has longitudinally opposite ends 181 and laterally opposite side edges 183, and is disposed below the central opening 176 of the inner layer 140, e.g., in the crotch region 46 of the garment 40, to receive body exudates that pass through the inner layer. The absorbent assembly 160 is suitably sized in length to be shorter than the overall length of the garment 40 but to otherwise extend longitudinally at least up to and more suitably beyond the longitudinally inner ends 162A, 164A of the front and back waist panels 162, 164 e.g., sized in length at least equal to and more suitably longer than the central opening 176 of the inner layer 140. The absorbent assembly 160 is also suitably sized in width to be slightly narrower than the overall width of the garment 40 at the crotch region 46, but to otherwise extend laterally at least up to and more suitably laterally outward of the laterally inboard edges 171 of the side panels 168 e.g., sized as wide as and more suitably wider than the central opening of the inner layer. It is understood, however, that the absorbent assembly 160 may extend longitudinally to the longitudinal ends 136 of the garment 40 and/or laterally to the side edges 134 of the diaper without departing from the scope of this invention.

The absorbent structure 155 of the absorbent assembly 160 may suitably comprise a, matrix of hydrophilic fibers, such as a web of cellulosic fluff, and may optionally further comprise a high-absorbency material commonly known as superabsorbent material. For example, see U.S. Ser. No. 10/954536, previously incorporated by reference. The absorbent assembly 160 may further include a surge management layer 161 positioned between the topsheet and the absorbent structure 155 to quickly take in and efficiently distribute liquid exudates to the absorbent structure 155.

The outer layer 148 is suitably extensible in at least the lateral direction. The outer layer 148 is also suitably soft-feeling, compliant and “breathable” or vapor permeable material. As an example, an extensible outer layer 148 can be composed of a necked fabric, a creped fabric, a micro-pleated fabric, polymer films or the like, as well as combinations thereof. The fabrics may be woven or nonwoven materials, such as spunbond fabrics.

Alternatively, the outer layer 148 of the garment 40 may be suitably constructed to be liquid impermeable. For example, in one embodiment the outer layer 148 may be comprised of a thin plastic film or other flexible liquid-impermeable material.

The outer layer 148 of the garment 40 is preferably formed from a stretchable material so as to be extensible or even elastic to facilitate stretching of the outer layer along with the inner layer 140 of the garment 40 as well as the side panels 168 where the side panels are secured to the outer layer. When the outer layer 148 is formed of a stretchable material, the outer layer is suitably capable of stretching in at least the lateral direction 32 and may additionally be stretchable in the longitudinal direction 30. It is contemplated that where the outer layer 148 is stretchable in both directions, it may be extensible in both directions, elastic in both directions, or extensible in one direction and elastic in the other direction.

The front and back waist panels 162, 164 are suitably secured to the outer layer 148 at their respective coterminous edges by adhesive bonding, ultrasonic bonding, thermal bonding or other suitable securement technique.

As seen in FIG. 4, the garment 40 may further comprise a pair of fastener tabs 190 (shown in parallel with longitudinal axis of the garment, but optionally set at an angle) and employed to secure the diaper about the waist of a wearer. Suitable fastener tabs 190 include hook-and-loop type fasteners, adhesive tape fasteners, buttons, pins, snaps, mushroom-and-loop fasteners, and the like.

When introducing elements of the present invention or the preferred aspects thereof, the articles “a”, “an”, “the”, and “said” are intended to mean that there are one or more of the elements. The terms “comprising,” “including”, and “having” are intended to be inclusive and mean that there may be additional elements other than the listed elements. As various changes could be made in the above constructions without departing from the scope of the invention, it is intended that all matter contained in the above description or illustrated in the accompanying drawings shall be interpreted as illustrative and not in a limiting sense.

What is claimed is:

1. A garment for wear about the lower torso, comprising:
a chassis defining a longitudinal axis, a rear waist region located at the rear of the garment and having opposing rear intermediate edges, and a rear waist edge having a rear waist width; an opposite front waist region located at the front of the garment and having opposing front intermediate edges, and a front waist edge having a front waist width that is narrower than the front edge; a crotch region defining a pair of opposed side edges having a lateral crotch width, a rear lateral hip width adjacent the rear waist region and a front lateral hip width adjacent the front waist region; and
a front pair of fasteners attached to the front waist region, wherein the front pair of fasteners are angled with respect to the longitudinal axis of the garment.

2. The garment of claim 1 wherein the front intermediate edges of the front waist region are angled so that the front waist width is less than the front hip width.

3. The garment of claim 1 further comprising a rear pair of fasteners, each one of the rear pair of fasteners located at the rear waist region.

4. The garment of claim 3 wherein the rear pair of fasteners are angled with respect to the longitudinal axis of the garment.

5. The garment of claim 4 wherein the front pair of fasteners form a side seam of a pant garment.

6. The garment of claim 4 wherein each of the rear pair of fasteners are at an angle of between about 14 and about 33 degrees.

7. The garment of claim 6 wherein the crotch width is between about 100 mm and about 177 mm.

8. The garment of claim 1 further comprising a front ear extending outwardly from the chassis at each of the front intermediate edges, and wherein the front pair of fasteners are located on the front ears.

9. The garment of claim 1 further comprising a rear ear extending outwardly from the chassis at each of the rear intermediate edges, and a rear pair of fasteners are located on the rear ears.

10. The garment of claim 1, wherein the garment is a pant and the front pair of fasteners are angled as determined by the following formula:
angle=arctangent ((a length of an ideal crotch side edge)−(a length of an actual crotch side edge))/2*(a length of the rear intermediate edge)
wherein the ideal crotch side edge is based on a body line fit for the garment.

11. The garment of claim 2, wherein the garment is a diaper and the front fasteners are angled as determined by the following formula:
angle=arctangent (( a length of an ideal crotch side edge)−(a length of an actual crotch side edge))/(a length of the rear intermediate edge)
wherein the ideal crotch side edge is based on a body line fit for the garment.

12. A method for determining an angle of a front pair of fasteners for a garment as defined by claim 1, comprising the steps of:
Determining an ideal body line for an average wearer of a predetermined garment size, comprising the steps of:
Measuring a length of an ideal crotch side edge;
Measuring a length of an actual crotch side edge of the garment; and
Calculating the angle from the length of the ideal crotch side edge and the length of the actual crotch side edge.

13. The method of claim 12 wherein the ideal body line is partially defined by a subgluteal crease and an inguinal crease of the wearer.

14. The method of claim 12 where an ideal body line is partially defined by a waist circumference of the wearer.

15. A diaper, comprising:
a chassis defining a longitudinal axis, a rear waist region located at the rear of the garment and having opposing rear intermediate edges, and a rear waist edge having a rear waist width; an opposite front waist region located at the front of the garment and having opposing front intermediate edges, and a front waist edge having a front waist width; a crotch region defining a pair of opposed side edges having a lateral crotch width, a rear lateral hip width adjacent the rear waist region and a front lateral hip width adjacent the front waist region;
a front pair of fasteners attached to the front waist region, wherein the front pair of fasteners are angled with respect to the longitudinal axis of the garment; and
wherein the front intermediate edges of the front waist region are angled so that the front waist width is less than the front hip width.

16. The diaper of claim 15 further comprising a rear pair of fasteners, each one of the rear pair of fasteners located at the rear waist region.

17. The diaper of claim 16 wherein the rear pair of fasteners are angled with respect to the longitudinal axis of the garment.
